(19) United States
(12) Patent Application Publication
(10) Pub. No.: US 2008/0248484 A1
(43) Pub. Date: Oct. 9, 2008

(54) METHOD FOR THE EARLY DETECTION OF PANCREATIC CANCER AND OTHER GASTROINTESTINAL DISEASE CONDITIONS

(76) Inventor: A. Robert Bauer, Summit Park, UT (US)

Correspondence Address: THORPE NORTH & WESTERN, LLP., P.O. Box 1219, SANDY, UT, 84091-1219, US

(21) Appl. No.: 12/143,605

(22) Filed: Jun. 20, 2008

Related U.S. Application Data
(63) Continuation of application No. 10/938,696, filed on Sep. 11, 2004.
(60) Provisional application No. 60/598,477, filed on Aug. 3, 2004.
(60) Provisional application No. 60/607,088, filed on Sep. 5, 2004.

(51) Int. Cl. C12Q 1/68

(52) U.S. Cl. 435/6

(57) ABSTRACT

The present invention uses peripheral blood monocyte-lymphocyte for the early diagnosis of pancreatic cancer, as well as other conditions of the pancreas and other organs. The peripheral blood lymphocytes recognize the new neoplasm in the pancreas, as well as disease processes in other organ systems. The evaluation of this specific recognition of the disease process by the peripheral blood monocyte-lymphocyte through gene microarray expression patterns constitute a successful method for the early detection of pancreatic cancer and other organ disease processes. This document describes the process used in this method of early diagnosis.

### RELATED APPLICATIONS

This application is a continuation of my copending application Ser. No. 10/938,696, filed Sep. 11, 2004, and entitled “The Discovery and a Method for the Early Detection of Pancreatic Cancer and Other Disease Conditions”, which claims the benefit of Provisional Patent Application Nos. 60/598,477, “Process for Early Identification of Cancer and Other Disease Conditions”, filed Aug. 3, 2004, and 60/607,088, “The Discovery and a Method for the Early Detection of Pancreatic Cancer and Other Disease Conditions”, filed Sep. 5, 2004.

### BACKGROUND

1. Field

This invention is in the field of methods for early diagnosis of pancreatic cancer and other disease conditions.

2. State of the Art

Pancreatic cancer is a deadly disease which has a mortality rate in the United States of more than 27,000 people a year, Lillemoe, K. D., C. J. Yeo, and J. L. Cameron, Pancreatic cancer: state-of-the-art care. CA Cancer J Clin, 2000. 50(4): p. 241-68. About 85% of those diagnosed with the disease have metastasis or spread of the disease beyond the pancreas and are almost impossible to cure with surgical resection, the only possible method of curing the disease at this time. If the growth is found sooner it may be resected with a much better hope of cure. Only about 15% of the newly diagnosed cases are resectable and the chances of a cure are usually 25% or less. Wiesenauer C. A. et al., Preoperative Predictors of Malignancy in Pancreatic Intraductal Papillary Mucinous Neoplasms. Arch. Surg; 2003 138: p 610-618; Ros, P. R. and K. J. Mortele, Imaging features of pancreatic neoplasms. Jbr-Btr, 2001. 84(6): p. 239-49; Ryu, B., et al., Relationships and differentially expressed genes among pancreatic cancers examined by large-scale serial analysis of gene expression. Cancer Res, 2002. 62(3): p. 819-26; Ito, M., et al., Molecular basis of T cell-mediated recognition of pancreatic cancer cells. Cancer Res, 2001. 61(5): p. 2038-46. Earlier diagnosis is the only hope of allowing earlier successful treatment at this time.

Since the dividing time of the pancreatic cancer cell is around 40 days, the cancer has been present for many months or years before it is detectable by present imaging and other diagnostic methods. Pathway markers have not as yet proved successful in the early diagnosis of pancreatic or other cancers with a high degree of specificity or sensitivity Lillemoe, K. D., C. J. Yeo, and J. L. Cameron, Pancreatic cancer: state-of-the-art care. CA Cancer J Clin, 2000. 50(4): p. 241-68; Rosty C, Goggins M., Early detection of pancreatic carcinoma. Hematol Oncol Clin North Am, 2002 16(1):37-52.

The dendritic cell or macrophage notes a new growth and tells the lymphocytes. The addition of major histocompatibility complexes helps identify the growth as part of the self. This includes T lymphocytes CD8 with HCS I and CD4 with HCS II and later B lymphocytes. Zeng, G., MHC Class II-Restricted Tumor Antigens Recognized by CD4+T Cells. New Strategies for Cancer Vaccine Design. J Immunother, 2001. 24(3): p. 195-204; Jonuleit, H., et al., Identification and functional characterization of human CD4(+)CD25(+) T cells with regulatory properties isolated from peripheral blood. J Exp Med, 2001. 193(11): p. 1285-94; Serbina N. V., Pamer E. G. Giving Credit Where Credit Is Due. Science, 2003, 301:1856-1857; and Baxevanis, C. N., et al., Tumor-specific CD4+T lymphocytes from cancer patients are required for optimal induction of cytotoxic T cells against the autologous tumor. J Immunol, 2000. 164(7): p. 3902-12. Tumor infiltrating lymphocytes, (TIL cells) often attack the new growth, but decrease in the area of the tumor later as tolerance develops. Ryschich, E., et al., Transformation of the microvascular system during multistage tumorigenesis. Int J Cancer, 2002. 97(6): p. 719-25. It has been shown that the CD4-CD25 T lymphocytes contribute to tolerance of developing cancer. Liyanage, U. K., et al., Prevalence of regulatory T cells is increased in peripheral blood and tumor microenvironment of patients with pancreas or breast adenocarcinoma. J Immunol, 2002. 169(5): p. 2756-61.

### SUMMARY OF THE INVENTION

The peripheral blood lymphocytes gene system recognizes and continues to react to the developing neoplasm. The developing changes in the tumor growth will be reflected in the statistically significant gene expression patterns in the peripheral blood lymphocytes compared to similar people of the same age and gender without the developing neoplasm (donor controls). This allows the early diagnosis of the developing disease.

The negatively selected CD8, CD4, CD4-CD25 T lymphocytes and B lymphocytes isolated from the peripheral blood of persons with pancreatic cancer and other disease conditions allows a more specific and focused early diagnosis.

### DETAILED DESCRIPTION OF THE INVENTION

The peripheral blood sample is obtained from the patient in the usual manner of obtaining venous blood from a peripheral vein, such as the anti-cubital vein of the arm. Usually 16 ml in two 8 ml tubes is drawn into a sterile RNase free vacumn tubes with a Ficoll type gradient and heparin. (Such as the BD Vacutainer CPT tubes with heparin.) These tubes are centrifuged at a centrifugal force of about 1500×g, using for example top of the tubes 17 cm from the center of the center post of the centrifuge, for 20 minutes at 2800 rpm at room temperature. The resulting ‘snow storm’ of monocyte-lymphocytes sits on top of the Ficoll gradient and below the clear plasma layer Approximately 2 ml of this monocyte-lymphocyte layer is aspirated with a sterile RNase free plastic Pasteur bulb tube and placed in a sterile RNase free 15 ml plastic tube with a screw top.

The cells in the aspirated sample are then washed. Approximately 13 ml of 1× PBS (phosphate buffered saline) solution made with RNase free water, is added to the plastic tube and centrifuged at 1300 rpm for 15 minutes. The same distance is used for the centrifuge as previously, 17 cm from the center of the center post of the centrifuge. This is done at room temperature. A small white pellet is found at the bottom of the centrifuged 15 ml plastic tube. The supernatant is gently poured from the tube without disturbing the pellet. The small remaining part of the supernatant is very gently aspirated from the tube, again not disturbing the pellet.

The cells in the pellet are now preserved in one of three ways. Method A. Two tubes with the pellets are used and 350 μg added of a B-ME (B-Mercapthanol) preservative. (Such as 10 μl of B-ME in 1 ml of Buffer RLT from the Quiagen RNeasy Mini Protect Kit.) Mild vortexing of the lysate with the pellets in the tube is gently done, holding the tube to the side of the rim of the vortexing machine. Allow the cells to be lysated for five or more minutes and draw back and forth through a sterile Rnase free #18 needle and 1 ml sterile Rnase free syringe five times gently. This amount from the two pellets in the two tubes is then transferred to one 1.5 ml Eppendorff sterile RNase free tube. This may then be stored at −80° C. or continued to be processed to total RNA (tRNA). Method B, The sample may instead be placed in a DMSO (dimethyl sulphoxide) solution made up of 500 μl of DMSO, 500 μl of the patient's own serum and 41 of RPMI 1640 which is mixed and then 1 ml added to the pellet at the bottom of the 15 ml plastic tube and gently vortex. This may then be slowly frozen to −80° C. for storage or immediately processed to total RNA (tRNA). If it is stored at −80° C. then it should be melted slowly to 37° C. before processing to total RNA. This procedure allows the cells to be negatively selected to lymphocyte subsets of CD8, CD4, CD4-CD25 and B lymphocytes which are then processed to aRNA or to cDNA as described below for microarray pattern recognition. Method C. 100 μl of RNlater (from Qiagen RNeasy Mini Protect Kit) may instead be added to the washed pellet as a preservative to stop enzyme degradation. This is thought to be a high salt solution and the cells in this solution may not be effectively negatively selected for subset analysis. The patterns in this method (Method C) are of the total monocyte-lymphocyte gene expression reaction to the neoplasm.

If the B-ME buffered method of lysate of two tubes of pellets (Method A) is used for further processing to total RNA, an equal amount of 70% ethanol made from pure absolute alcohol with 30% of RNase free non-DEPCA treated sterile water added to the alcohol, is added to the cell containing lysate in the Eppendorff tube. This is gently mixed and then in 700 μl amounts added to a silica gel column. (Such as that supplied by Qiagen in their Mini Protect Kit.) This is then centrifuged at 10,000 rpm (approximately 9,000.g.) for one minute and the flow through discarded. The ethanol bounded total RNA with higher amount of messenger RNA (mRNA) is bound to the silica gel membrane which is then washed and eluted in sterile RNase free water. In more detail, the remaining lysate in the Eppendorff tube is transferred in 700 μl or less volume to the silica column and centrifuged in a microcentrifuge again for one minute at the same speed, 10,000 rpm. The flow through is discarded and 350 μl of a wash solution. (Such as that from the Qiagen RNeasy Mini Protect Kit). Is placed on the column and again centrifuged for one minute at 10,000 rpm. Following this add 10 μl of DNase1 stock solution from an RNase Free DNAse Set (Introvirogen) to 70 μl RDD buffer. This eliminates the remaining small amount of DNA leaving the enriched mRNA. Mix gently by inverting and add gently to the silica gel column. Let stand for 15 minutes then wash again with 350 μl of a wash solution, microcentrifuging for one minute at 10,000 rpm. Discard the flow through.

Pipette 500 μl of Buffer RPE from the Qiagen Kit to the column and centrifuge for one minute at 10,000 rpm using the same collection tube. Discard the flow through. Pipette another 500 μl of RPE Buffer solution (again to wash the column with ethanol) to the column with a new collection tube and centrifuge again for one minute at 10,000 rpm in a microcentrifuge. If the column is not totally dry, discard the flow through and recentrifuge at 16,000 rpm for one minute. Do not do this last step, if the column is dry.

Transfer the dry silica gel column to a new 1.5 m RNase free collection tube and pipette 30 μl of RNase free sterile water directly onto the silica gel membrane, holding the pipette only one or two millimeters above the membrane. Microcentrifuge the column at 10,000 rpm for one minute. This gives 30 μl of total RNA (tRNA). One may then OD (optical density with UV spectrophotometry) one μl of this, with or without dilution, to determine the concentration or quantity of total RNA (aRNA). One may also run a gel to be sure the bands indicate no degradation of the total RNA.

After the total RNA ( tRNA) is measured for concentration by OD, 3 μg is used for the T7 method of linear amplification. This may very from 500 nanograms to 5 μg, but 3 μg is the preferred amount. The solution is diluted to 10 μl volume, if dilution is necessary, with sterile RNase free water. A 10 fold amplification of the original quantity is desired. Actually the amplification of the messenger RNA to amplified anti-sense RNA (aRNA) is much greater, since the percentage of polyadenylated RNA (AAA messenger RNA) is very small compared to the amount of total RNA in the sample.

1 μg of the first strand synthesis promoter primer is added to the 10 μl of specimen after first thawing, mixing and briefly spinning the primer from the −20° C. frost free refrigerator. This 1 μg is carefully mixed and spun. Then, the specimen is placed in a thermal cycler for incubation at 65° C. for 5 minutes and cooled to 4° C. for denaturing and annealing. This denatures the total RNA and anneals the primer. (If experimentally one is using the Arcturus Kit [Arcturus RiboAmp RNA Amplification Kit #KIT0201 then Primer A is used in this step, according to their directions.)

First strand synthesis solutions (including dNTP, polymerase and buffers for pH) are thawed, mixed and spun for 2 seconds. They are then added to the specimen, stirred, mixed and briefly spun. This specimen is then placed in a thermal cycler for 60 minutes at 42° C. and cooled to 4° C. (If experimentally one is using the Arcturus Kit one mixes 7 μl of 1^{st} Strand Master Mix, then 2 μl of the 1^{st} Strand Enzyme Mix, giving a total of 20 μl.)

After the first strand synthesis a nuclease mix is thawed, mixed and spun briefly. 2 μl is added to the specimen which is briefly spun after being cooled to 4° C. after the last step. The nuclease mix is added to the specimen and spun briefly with a small lab bench centrifuge. This leaves the first strand synthesized specimen. It is then placed in the thermal cycler for 20 minutes at 37° C., 95° C. for 5 minutes and cooled to 4° C. destroying the nuclease after its effect. Spun down briefly. (Experimentally with the Arcturus Kit, one uses the 1^{St} Strand Nuclease Mix as directed.)

1 μl of the second primer is added to this mix after cooling to 4° C. for two or more minutes and spinning briefly. The specimen is then placed in a thermal cycler for 2 minutes at 95° C. and cooled to 4° C. (If experimentally using the Arcturus Kit, Primer B is used for this step of denaturing.)

2^{nd} strand synthesis solutions (dNTP, polymerase and buffers) are thawed, mixed and briefly spun. Then, added to the specimen. This is placed in a thermal cycler for 10 minutes at 25° C., 37° C. for 20 minutes and 70° C. for 5 minutes. Then, cooled to 4° C. ( If experimentally one is using the Arcturus Kit 29 μl of 2^{nd} Strand Master Mix is used and then 1 μl of the 2^{nd} Strand Enzyme Mix, giving a total of 53 μl together with the specimen.)

Purification is then carried out by binding cDNA resulting from the previous steps to a column and washing the column with wash buffers of alcohol. Then, the DNA is eluted and taken to the next step. (If one experimentally is using the Arcturus Kit, 250 μl of DNA Binding Buffer is added to the DNA/RNA Purification Column in a collection tube and after several minutes at room temperature centrifuged at full in a microcentrifuge, 16,000×g for one minute to prepare and wet the column, as per directions. Discard the flow through. Then, 200 μl of the DNA Binding Buffer is added to the 2^{nd} strand synthesis specimen very carefully with gentle thorough mixing and pipetted to the previously coated purification column. It is then centrifuged at 100×g for two minutes and then 10,000×g for 1 minute. Discard the flow through. The column is then washed with 250 μl of the Arcturus DNA Wash Buffer to the column and centrifuged at 16,000×g for one minute. If the column is very dry transfer it to a 0.5 ml microcentrifuge tube and place 16 μl of elution buffer onto the center of the column from about 1 to 2 millimeters above the column. Allow to stand for two minutes, then microcentrifuge at 1,000×g for one minute, then 16,000×g for one minute. The flow through contains the purified cDNA.)

The process should be continued immediately by adding transcription solutions (buffers, dNTP and polymerase) and incubating in a thermal cycler with a heated lid at 42° C. for 4 hours then cooling to 4° C. (If one is experimentally using the Arcturus Kit, thaw, mix and spin the IVT Reaction solutions. Then, add 8 μl of the IVT Buffer, followed by 12 μl of the IVT Master Mix and 4 μl of the IVT Enzyme Mix, spinning after thoroughly mixing these together with the 16 μl of cDNA specimen. Place for 4 hours in the thermal cycler as above at 42° C.)

After the above step add a DNA nuclease mix to leave only amplified anti-sense RNA (aRNA) to the 4° C. cooled specimen and place in a thermal cycler for 15 minutes at 37° C. Again, cooling to 4° C. (If experimentally using the Arcturus Kit use 2 μl of DNase Mix thaw, mix and add to specimen, mixing and spinning prior to thermal cycler.)

Follow the above step with purification of the amplified anti-sense RNA (aRNA) with adherence to a wetted or prepared column and washing with an ethyl alcohol buffered solution. (If experimentally using the Arcturus Kit add 250 μl of RNA Binding Buffer to a DNA/RNA Purification Column, allowing this to stand for several minutes. Then spin the column at 16,000×g in a lab bench microcentrifuge for one minute. Discard the flow through. Next, add 200 μl of this buffer to the specimen gently mixing it and pipette it to the purification column. Microcentrifuge at 100×g for two minutes and 10,000×g for one minute. Wash with 200 μl of RNA Wash Buffer added to the column and microcentrifuge at 10,000×g for one minute. Discard the flow through. Again, add 200 μl of RNA Wash Buffer to the column and centrifuge at 16,000μg to two minutes. Discard the flow through. Be sure the column is very dry. If it is not dry, then centrifuge at 16,000×g for one minute again.)

To the dry column placed in a 0.5 ml microcentrifuge tube add 30 μl of elution solution and allow to stand for two minutes, placing the solution very carefully just above the center of the column, one to two millimeters above the column. After the two minutes, microcentrifuge the column at 1,000×g for one minute, then 16,000×g for one minute. The 30 μl of flow through contains the aRNA. (If one is experimentally using the Arcturus Kit, use RNA Elution Buffer to elute the column.)

The aRNA may now be stored at −80° C. One may measure the concentration with OD, optical density, and run a gel to check for aRNA degradation.

Following the above steps the aRNA may be checked with a bio-analyzer and microarrayed for gene expression. The gene expression patterns will then be analyzed with advanced software (such as R software) to determine the statistically significant expression of the pancreatic cancer and other disease conditions compared to the normal expected patterns of non-diseased control samples.

This method may be modified to increase the availability and reduce the laboratory time and cost of the test with the use of direct linear amplification of smaller amounts of total RNA to cDNA for direct attachment of dyes for microarray with different or hybrid promoters and primers (such as with the NuGene method). Also, this discovery may be enhanced by use with newer microfluid chips. Even more focused gene patterns may be evaluated with negatively selected combinations of subsets of the T and B lymphocytes for patterns of gene expression of early developing tumors, allowing early resection or destruction of the tumor before metastatic spread of the subsequent cancer

This method will give the patterns needed for the early diagnosis of the pancreatic cancer and other conditions. This method describes one useful method of accomplishing the discovery claimed in this patent application of using the peripheral blood monocyte-lymphocytes for the early diagnosis of pancreatic cancer and other disease conditions.

I claim:

1. A method for identifying gene expression patterns useful in detecting a particular selected disease condition in a patient to be screened for the particular disease condition, such gene expression patterns being determined for genes being present in a patient's peripheral blood monocyte-lymphocytes, comprising the steps of:
obtaining a sample of peripheral blood monocyte-lymphocytes from a person known to be suffering from the particular disease condition for which it is desired to be able to screen the patient;
mixing the sample of peripheral blood with heparin as it is taken from the person;
processing the sample of peripheral blood monocyte-lymphocytes to allow statistically significant gene expression patterns for the sample peripheral blood monocyte-lymphocytes to be obtained;
obtaining a gene expression pattern showing the statistically significantly expressed genes in the sample peripheral blood monocyte-lymphocytes; and
comparing the gene expression pattern so obtained with a gene expression pattern for peripheral blood monocyte-lymphocytes from a person known not to be suffering from the disease condition for which the patient is desired to be screened, the genes showing statistically significant differences between the compared gene expression patterns indicating a gene expression patterns useful in detecting the particular diseased condition in the patient.

2. A method for identifying gene expression patterns useful in detecting a particular selected disease condition in a patient to be screened for the particular disease condition, according to claim 1, wherein the step of processing the sample of peripheral blood monocyte-lymphocytes to allow the sample peripheral blood monocyte-lymphocytes includes the steps of processing the peripheral blood monocyte-lymphocytes to total RNA, and obtaining amplified aRNA or cDNA from the total RNA.

3. A method for identifying gene expression patterns useful in detecting a particular selected disease condition in a patient to be screened for the particular disease condition, according to claim 2, wherein the total RNA includes polyadenylated messenger RNA, and the step of obtaining amplified aRNA or cDNA from the total RNA obtains the amplified aRNA or cDNA from the polyadenylated messenger RNA.

4. A method for identifying gene expression patterns useful in detecting a particular selected disease condition in a patient to be screened for the particular disease condition, according to claim 2, wherein the step of obtaining a gene expression pattern showing the statistically significantly expressed genes in the sample peripheral blood monocyte-lymphocytes obtains a gene expression pattern for the amplified anti-senseaRNA or cDNA.

5. A method for identifying gene expression patterns useful in detecting a particular selected disease condition in a patient to be screened for the particular disease condition, according to claim 4, wherein the step of obtaining a gene expression pattern showing the statistically significantly expressed genes in the sample peripheral blood monocyte-lymphocytes obtains a gene expression microarray pattern for the amplified anti-sense aRNA or cDNA.

6. A method for identifying gene expression patterns useful in detecting a particular selected disease condition in a patient to be screened for the particular disease condition, according to claim 1, wherein the step of obtaining a gene expression pattern showing the statistically significantly expressed genes in the sample peripheral blood monocyte-lymphocytes obtains a gene expression microarray pattern for the genes.

7. A method for identifying gene expression patterns useful in detecting a particular selected disease condition in a patient to be screened for the particular disease condition, according to claim 1, wherein the gene expression pattern for peripheral blood monocyte-lymphocytes from a person known not to be suffering from the disease condition for which the patient is desired to be screened which is compared with the gene expression pattern from the person known to be suffering from the particular disease condition, is obtained by:
obtaining a sample of peripheral blood monocyte-lymphocytes from the person known not to be suffering from the particular disease condition for which it is desired to be able to screen the patient;
mixing the sample of peripheral blood with heparin as it is taken from the person;
processing this sample of peripheral blood monocyte-lymphocytes to allow statistically significant gene expression patterns for this sample peripheral blood monocyte-lymphocytes; and
obtaining a gene expression pattern showing the statistically significantly expressed genes in this sample of peripheral blood monocyte-lymphocytes.

8. A method for identifying gene expression patterns useful in detecting a particular selected disease condition in a patient to be screened for the particular disease condition, according to claim 1, wherein the particular selected disease condition to be screened for is a gastrointestinal disease.

9. A method for identifying gene expression patterns useful in detecting a particular selected disease condition in a patient to be screened for the particular disease condition, according to claim 8, wherein the particular selected disease condition to be screened for is pancreatic cancer.

10. A method of obtaining a gene expression pattern for peripheral blood monocyte-lymphocytes from a living body, comprising the steps of:
obtaining a sample of peripheral blood from the living body;
separating and obtaining from the sample of peripheral blood a sample of sets of CD8, CD4, and CD4-CD25 T lymphocytes and B lymphocytes;
processing the sets of CD8, CD4, and CD4-CD25 T lymphocytes and B lymphocytes to allow determination of genes statistically significantly expressed in the sets of CD8, CD4, and CD4-CD25 T lymphocytes and B lymphocytes; and
obtaining a gene expression pattern of the statistically significantly expressed genes in the sets of CD8, CD4, and CD4-CD25 T lymphocytes and B lymphocytes.

11. A method of obtaining a gene expression pattern for peripheral blood monocyte-lymphocytes from a living body, according to claim 10, wherein the step of separating and obtaining a sample of sets of CD8, CD4, and CD4-CD25 T lymphocytes and B lymphocytes obtains the sets of CD8, CD4, and CD4-CD25 T lymphocytes and B lymphocytes through negative selection of the cells which are then processed to total RNA with amplification of polyadenylated messenger RNA to amplified anti-sense aRNA or to cDNA.

12. A method of obtaining a gene expression pattern for peripheral blood monocyte-lymphocytes from a living body, according to claim 11, wherein the step of obtaining a gene expression pattern of the statistically significantly expressed genes in the sets of CD8, CD4, and CD4-CD25 T lymphocytes and B lymphocytes obtains a gene expression pattern for the amplified anti-sense aRNA or cDNA.

13. A method of obtaining a gene expression pattern for peripheral blood monocyte-lymphocytes from a living body, according to claim 12, wherein the step of obtaining a gene expression pattern of the statistically significantly expressed genes in the sets of CD8, CD4, and CD4-CD25 T lymphocytes and B lymphocytes obtains a gene expression microarray pattern for the amplified anti-sense aRNA or cDNA.

14. A method of obtaining a gene expression pattern for peripheral blood monocyte-lymphocytes from a living body, according to claim 10, wherein the step of processing the sets of CD8, CD4, and CD4-CD25 T lymphocytes and B lymphocytes to allow determination of genes statistically significantly expressed in the sets of CD8, CD4, and CD4-CD25 T lymphocytes and B lymphocytes includes the steps of processing the sets of CD8, CD4, and CD4-CD25 T lymphocytes and B lymphocytes to total RNA, and obtaining amplified aRNA or cDNA from the total RNA.

15. A method of obtaining a gene expression pattern for peripheral blood monocyte-lymphocytes from a living body, according to claim 14, wherein the total RNA includes polyadenylated messenger RNA, and the step of obtaining amplified aRNA or cDNA from the total RNA obtains the amplified aRNA or cDNA from the polyadenylated messenger RNA.

16. A method of obtaining a gene expression pattern for peripheral blood monocyte-lymphocytes from a living body, according to claim 15, wherein the step of obtaining a gene expression pattern of the statistically significantly expressed genes in the sets of CD8, CD4, and CD4-CD25 T lymphocytes and B lymphocytes obtains a gene expression pattern for the amplified anti-sense aRNA or cDNA.

17. A method of obtaining a gene expression pattern for peripheral blood monocyte-lymphocytes from a living body, according to claim 10, wherein the step of obtaining a gene expression pattern of the statistically significantly expressed genes in the sets of CD8, CD4, and CD4-CD25 T lymphocytes and B lymphocytes obtains a gene expression microarray pattern for amplified anti-sense aRNA or cDNA.

18. A method of obtaining a gene expression pattern for peripheral blood monocyte-lymphocytes from a living body, comprising the steps of:
obtaining a sample of peripheral blood from the living body and mixing the sample with heparin as it is taken from the living body; and
processing the sample of peripheral blood monocyte-lymphocytes to allow statistically significant gene expression patterns for the sample peripheral blood monocyte-lymphocytes to be obtained.

19. A method of obtaining a gene expression pattern for peripheral blood monocyte-lymphocytes from a living body, according to claim 18, additionally including the step of centrifuging the blood and heparin mixture to concentrate the monocyte-lymphocytes, and removing the concentrated monocyte-lymphocytes for further processing.
